(19) United States
(12) Patent Application Publication
(10) Pub. No.: US 2026/0328787 A1
(43) Pub. Date: Oct. 22, 2026

(54) MEANS AND METHODS FOR DETERMING FIBRINOGEN USING AN ASSAY BASED ON A COMPETITIVE MECHANISM

(71) Applicant: Roche Diagnostics Operations, Inc., Indianapolis, IN (US)

(72) Inventors: Carina HORN, Biblis (DE); Sascha MEYER DOS SANTOS, Portimão (PT); Dagmar SCHUMACHER, Indianapolis, IN (US)

(21) Appl. No.: 19/694,649

(22) Filed: Jun. 1, 2026

Related U.S. Application Data
(63) Continuation of application No. PCT/EP2024/084115, filed on Nov. 29, 2024.

(30) Foreign Application Priority Data
Dec. 1, 2023 (EP) 23213719.0

(51) Int. Cl. G01N 33/86

(52) U.S. Cl. CPC G01N 33/86; C12Y 304/21074; G01N 2333/75; G01N 2470/12; G01N 2800/224

(57) ABSTRACT

The present disclosure provides methods for determining fibrinogen in a sample, which include the steps of (a) contacting a fibrinogen binding agent, said fibrinogen binding agent comprising a first molecule which is capable of specifically binding fibrinogen and, reversibly bound to the said first molecule, a second molecule which is capable of specifically binding the first molecule, wherein the affinity of said second molecule for the first molecule is lower than the affinity of fibrinogen for said first molecule, with a sample suspected to comprise fibrinogen for a time and under conditions which allow for specific binding of fibrinogen to said first molecule, whereby the second molecule is released from said first molecule and fibrinogen is specifically bound by the first molecule, (b) determining said second molecule released from said first molecule, and (c) determining fibrinogen in the sample based on the released second molecule.

### CROSS-REFERENCE TO RELATED APPLICATIONS

This application is a continuation of International Patent Application No. PCT/EP2024/084115, filed Nov. 29, 2024, which claims the benefit of priority to European Patent Application No. 23213719.0, filed Dec. 1, 2023, the disclosures of which are herein incorporated by reference in their entirety.

### TECHNICAL FIELD

The present disclosure relates to methods and kits for detecting fibrinogen, and diagnostic assays based on the same.

### BACKGROUND

Fibrinogen is one of the key coagulation factors and required for proper coagulation and clot formation. It is physiologically found in the blood in a range between 1.5 to 3.0 mg/ml. During coagulation, fibrinogen is proteolytically activated by thrombin (factor IIa) resulting in the formation of fibrin and fibrinopeptides A and B (N- and C-terminal fragments of fibrinogen). The fibrin monomers interact to form fibrin polymers, which are cross-linked to form cross-linked in order to generate a clot. The fibrinogen level in a subject is a pivotal clinical factor for the coagulation capacity of the subject and, hence, a key factor for emergency treatments and support. Various inherited or acquired disorders of coagulation are known. Further, diseases may also affect the coagulation capacity.

Current tests aiming at determining the coagulation capacity of a subject trigger the coagulation cascade at different levels. The gold-standard nowadays is the so-called “Clauss-assay” (Mackie et al, Thromb Haemost. 2002 June; 87 (6): 997-1005). The performance of this test is time-consuming and requires special technical expertise. Calibration is also not straight forward. It is, however, not possible to directly determine the fibrinogen level using said test. Immunoassay-based determination of fibrinogen is, in principle, possible. However, such immunoassays are also time-consuming and require special equipment.

The fibrinogen-cleaving properties of batroxobin have been known since mid-last century. In 1979, Heimann et al. proposed a diagnostic assay employing batroxobin to measure a heparin-insensitive, fibrinogen-based clotting time in addition to thrombin time (Heimann 1979, J. Clin. Chem. Clin. Biochem. 17 (6) 369-72). Cuvette-based assays for fibrinogen level determination based on snake toxins are also commercially available (see, e.g., WO2019/068940A1). These assays use a competitive approach in which a chromogenic substrate is cleaved by batroxobin, unless batroxobin is bound by the fibrinogen comprised in the sample. Thus, an inversely proportional signal is generated in said assays by proteolytic cleavage of the competitive substrate. However, a disadvantage of this approach is the inversely proportional relationship between the fibrinogen level and the substrate signal, since this could influence sensitivity negatively in the relevant low fibrinogen content.

Other point-of-care devices for testing fibrinogen levels that are commercially available, such as TEG or ROTEM, are not fibrinogen-specific, not portable and need a relatively high turnaround time (10-15 min). The respective TEG test (TEG Functional Fibrinogen) employs extrinsic coagulation activators and a GPIIb/IIIa blocker to minimize platelet influence in whole blood samples while the respective ROTEM (FIBTEM) test also uses extrinsic activation and cytochalasin D to reduce platelet influence. Both devices measure clot formation by mechanical clot detection systems.

### BRIEF SUMMARY

The following passages introduce a selection of concepts in a simplified form that are further described below in the Detailed Description. This summary of selected aspects is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

Trauma patients or patients undergoing large surgical interventions with major bleeding risks, e.g., cardiac surgery or larger abdominal surgeries, or women post partem, are at risk of bleeding complications mainly due to a strong decrease in fibrinogen levels. A fast, accurate point-of-care test for determining the clottable fibrinogen would help to peri- or postoperatively identify patients at risk and also control therapeutic intervention with, e.g., fibrinogen concentrate or fresh frozen plasma. The present disclosure provides methods for assaying fibrinogen in a biological sample and kits based on the same, which may be quickly deployed an/or implemented as a point-of-care test.

In a first general aspect, the disclosure provides a method for a fibrinogen in a sample comprising the steps of: (a) contacting a fibrinogen binding agent, said fibrinogen binding agent comprising a first molecule which is capable of specifically binding fibrinogen and, reversibly bound to the said first molecule, a second molecule which is capable of specifically binding the first molecule, wherein the affinity of said second molecule for the first molecule is lower than the affinity of fibrinogen for said first molecule, with a sample suspected to comprise fibrinogen for a time and under conditions which allow for specific binding of fibrinogen to said first molecule, whereby the second molecule is released from said first molecule and fibrinogen is specifically bound by the first molecule; (b) determining said second molecule released from said first molecule; and (c) determining fibrinogen in the sample based on the released second molecule.

In some aspects, the sample is a body fluid sample, optionally, a blood plasma sample or a whole blood sample.

In some aspects, the first molecule is an antibody or an aptamer

In some aspects, the first molecule is an enzyme. In some aspects, the enzyme upon specific binding to said fibrinogen (i) does not release fibrinogen or (ii) does not cleave fibrinogen and release fibrinogen cleavage product. In some aspects, the enzyme is a serine endopeptidase (E.C.3.4.21), optionally, a thrombin-like serine protease. In some aspects, the enzyme is a thrombin-like serine protease having venombin A activity, optionally, a batroxobin.

In some aspects, the second molecule comprises a tripeptide capable of specifically binding to the fibrinogen binding site of the first molecule.

In some aspects, the tripeptide is selected from the group consisting of N-terminus-Ile-Pro-Arg-C-terminus, N-terminus-Tos-Gly-Pro-Arg-N-terminus, N-terminus-Pro-Phe-Arg-C-terminus and N-terminus-Tos-Gly-Pro-Lys-C-terminus. In some aspects, the second molecule is N-alpha acetyl-lysine-4-nitroanilide (CAS No. 50931-35-6).

In some aspects, the second molecule is selected from the group consisting of N-alpha-substituted lysines, 4-amidinophenyl-alpha-aminobutyric acid amides, modified aminoiminomethylamino piperidinecarboxylic acids with bulky hydrophobic sidechains, modified argatroban, and 3- or 4-substituted benzamidines.

In some aspects, the second molecule comprises a detectable label. In some aspects, the detectable label is an optically detectable label or an electrochemically detectable label.

In some aspects, the second molecule comprises a label that can be enzymatically activated.

In a second general aspect, the disclosure provides a method for assessing coagulation defects or disorders in a subject comprising the steps of: (aa) determining fibrinogen in a sample of said subject according to any method for determining fibrinogen in a sample described herein; (bb) comparing the determined fibrinogen to a reference; and (cc) assessing coagulation defects or disorders in said subject based on said comparison.

In a third general aspect, the disclosure provides a device for determining fibrinogen in a sample comprising: (A) an analyzing unit comprising (i) a reaction zone comprising a fibrinogen binding agent, said fibrinogen binding agent comprising a first molecule which is capable of specifically binding fibrinogen and, reversibly bound to the said first molecule, a second molecule which is capable of specifically binding the first molecule, wherein the affinity of said second molecule for the first molecule is lower than the affinity of fibrinogen for said first molecule, wherein said reaction zone is configured for contacting the fibrinogen binding agent with a sample suspected to comprise fibrinogen for a time and under conditions which allow for specific binding of fibrinogen to said first molecule whereby the second molecule is released from the first molecule and fibrinogen is specifically bound by the first molecule; and (ii) a detector which is capable of determining the second molecule released from the fibrinogen binding agent; and (B) an evaluation unit which is capable of determining fibrinogen in the sample based on the released second molecule.

In a fourth general aspect, the disclosure provides a kit for determining fibrinogen in a sample, comprising a fibrinogen binding agent, said fibrinogen binding agent comprising a first molecule which is capable of specifically binding fibrinogen and, reversibly bound to the said first molecule, a second molecule which is capable of specifically binding the first molecule, wherein the affinity of said second molecule for the first molecule is lower than the affinity of fibrinogen for said first molecule

Advantageous embodiments which might be realized in an isolated fashion or in any arbitrary combinations are listed in the dependent claims as well as throughout the specification.

### BRIEF DESCRIPTION OF THE DRAWINGS

Various features and advantages of aspects of the present disclosure will be more fully understood from the following detailed description of illustrative embodiments taken in conjunction with the accompanying drawings in which:

FIG. 1 is a schematic overview of the measurement principle of the fibrinogen assay; fibrinogen present in a sample outcompetes the tripeptide substrate bound to Batroxobin, releasing the tripeptide substrate.

FIG. 2 is a graph showing a comparison of catalytic activities of recombinant (r), native (n) batroxobin and thrombin on various synthetic substrates (2).

FIG. 3 is a graph showing photometric monitoring of cleavage of the serine protease substrates B7632 (400 μM) and S-2288 (400 μM) by batroxobin (7 U/mL) and thrombin (0.1 U/mL), respectively. The graphs show mean values of duplicate measurements. Photometer: Tecan Sunrise microplate reader; conditions: 37 °C, Tris-HCl buffer pH 8.0, reaction volume 70 μL.

FIG. 4A and FIG. 4C show photometric measurements at 37 °C after addition of thrombin (0.1 U/mL) and previous preincubation of S-2288 (FIG. 4A) or B7632 (FIG. 4C) with batroxobin (7 U/mL), pefabloc FG (2.3 mM) and serial dilutions of fibrinogen plasma (Siemens Dade Ci-Trol 1). FIG. 4B and FIG. 4D show absorbance level plotted against the fibrinogen content in the sample at 10 min after the addition of thrombin for the reactions in A and C for S-2288 (FIG. 4B) and B7632 (FIG. 4D) as well as a polynomic fit including the coefficient of determination. The graphs in FIG. 4A and FIG. 4C show mean values of n=2-3 replicates. Photometer: Tecan Sunrise microplate reader; conditions: 37 °C, plasma Siemens Dade Ci-Trol 1 and serial dilutions with 0.9% NaCl, batroxobin 7 U/mL, pefabloc FG 2.3 mM, substrate 200 μM, reaction volume 70 μL.

FIG. 5A is a graph showing reduction of the nitro group of serial dilutions of the substrate S-2288 by cyclic voltammetry (CV) at 20 mV/s, 0 to −800 mV at room temperature. Buffer: KH₂PO₄ 95 mM, K₂HPO₄ 174 mM, NaCl 154 mM, Geropon T-77 0.05%, pH 7.0, working electrode: Au (1 cm²), counter electrode: Ag/AgCl (1 cm²). The graph shows mean values from two measurements for each concentration. FIG. 5B is a graph showing a dose dependent reduction peak can be observed between −600 and −650 mV. When the peak currents are plotted against the concentrations of S-2288 in the sample, a linear correlation with a correlation coefficient of 0.997 is found.

FIG. 6 is a diagram showing potential electrochemically active groups for replacement of nitrosoaniline in substrate S-2288.

FIG. 7 is a graph showing photometric monitoring of cleavage of the serine protease substrates CTHLys (Tos-Gly-Pro-Lys-pNA, 400 μM) and LyspNA (Ac-Lys-pNA, 400 μM) by batroxobin (0.5 U/mL) and plasmin (1 U/mL), respectively.

### DETAILED DESCRIPTION

The present disclosure relates to methods, assays, devices, and kits for measuring fibrinogen in a sample (e.g., blood or another biological fluid or fraction thereof). The inventive embodiments described herein enable accurate and easily deployable point-of-care devices to assay fibrinogen in a clinical setting, promoting improved patient care and therapeutic outcomes.

It is to be understood that as used in the specification and in the claims, “a” or “an” can mean one or more, depending upon the context in which it is used. Thus, for example, reference to “an” item cell” can mean that at least one item can be utilized.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. The term “comprising” also encompasses embodiments where only the items referred to are present, i.e. it has a limiting meaning in the sense of “consisting of”, or not.

Further, as used in the following, the terms “particularly”, “more particularly”, “typically”, and “more typically” or similar terms are used in conjunction with additional/alternative features, without restricting alternative possibilities. Thus, features introduced by these terms are additional/alternative features and are not intended to restrict the scope of the claims in any way. The present methods (devices, assays, etc.) may, as the skilled person will recognize, be performed by using alternative features. Similarly, features introduced by “in an embodiment of the invention” or similar expressions are intended to be additional/alternative features, without any restriction regarding alternative embodiments of the invention, without any restrictions regarding the scope of the invention and without any restriction regarding the possibility of combining the features introduced in such way with other additional/alternative or non-additional/alternative features of the invention.

Further, it will be understood that the term “at least one” as used herein means that one or more of the items referred to following the term may be used in accordance with the present disclosure. For example, if the term indicates that at least one sampling unit shall be used this may be understood as one sampling unit or more than one sampling units, e.g., two, three, four, five or any other number. Depending on the item the term refers to the skilled person understands as to what upper limit the term may refer, if any.

The term “about” as used herein means that with respect to any number recited after said term an interval accuracy exists within in which a technical effect can be achieved. Accordingly, about as referred to herein, preferably, refers to the precise numerical value or a range around said precise numerical value of +20%, preferably #15%, more preferably #10%, or even more preferably +5%.

Furthermore, the terms “first”, “second”, “third”, or “(a)”, “(b)”, “(c)”, “(d)” etc., and the like in the description and in the claims are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein. In case the terms “first”, “second”, “third” or “(a)”, “(b)”, “(c)”, “(d)”, “i”, “ii”, etc., relate to steps of a method or use or assay, there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that the methods, devices, assays and other inventive subject matter described herein is not limited to the particular methodology, protocols, reagents etc. described herein as these may vary. It is also to be understood that the terminology used herein is for the purpose of describing particular embodiments only, and is not intended to limit the scope of the present invention that will be limited solely by the appended claims. Unless defined otherwise, all technical and scientific terms used herein have the same meanings as commonly understood by one of ordinary skill in the art.

The methods described herein are typically performed ex vivo. The present methods may comprise the steps mentioned before, e.g., they may consist of those steps or may comprise additional steps. Moreover, the present methods may, typically, be assisted by automation where feasible. For example, the application and handling of samples may be assisted by robotics, while the determination of fibrinogen may be carried out by a data processing unit adapted for carrying out the necessary comparisons and/or calculations.

The term “determining” as used herein refers to qualitative and quantitative determination of the second molecule or fibrinogen, i.e. the term encompasses the determination of the presence or absence or the determination of the absolute or relative amount of said molecules.

Determining said second molecule as referred to in step (b) of the methods of the present disclosure may be carried out by any technique which allows for detecting the presence or absence or the amount of said second molecule upon its release from the first molecule. Suitable techniques depend on the molecular nature and the properties of the second molecule and are discussed elsewhere herein in more detail.

Determining fibrinogen as referred to herein is achieved by deducing or calculating the presence, absence or amount of fibrinogen from the presence, absence or amount of the second molecule. Further details are discussed elsewhere herein in more detail.

The term “fibrinogen” as used herein refers to a coagulation factor participating in the coagulation cascade. It is a glycoprotein complex, also known as factor I, made in the liver. Fibrinogen exists in numerous vertebrates. Fibrinogen is enzymatically cleaved and activated into fibrin by thrombin (factor IIa). Fibrin molecules are known to form blood clots in order to stop bleeding after, e.g., injury. In addition, fibrinogen mediates blood platelet and endothelial cell spreading, tissue fibroblast proliferation, capillary tube formation, and angiogenesis. Thereby, it is capable of promoting revascularization and wound healing.

The structure of fibrinogen is well known in the art. Typically, fibrinogen is a glycoprotein complex composed of different chains, i.e. an Aα chain, Bβ chain, and γ chain. Fibrinogen is a hexamer of two AαBβγ trimers which is glycosylated, hydroxylated, sulfated and phosphorylated in the mature stage. Mature fibrinogen has a molecular weight of about 340 kDa. It is typically found in human blood plasma in an amount of 1.5 to 3.0 mg/ml. During clot formation, fibrinogen is cleaved at the N-termini of the Aα chain and Bβ chain. As a result two fibrin strands are formed and two fibrinopeptides, i.e. fibrinopeptide A and B, are released. Said fibrin strands subsequently polymerize in order to form the clot. Amino acid sequences and structures of the fibrinogen chains are well known in the art and are described, typically, under UniProt accession numbers P02671 (human fibrinogen alpha chain), P02675 (human fibrinogen beta chain, and P02679 (human fibrinogen gamma chain) for human fibrinogen.

Fibrinogens may be dysfunctional or their level may be reduced in various congenital and acquired fibrinogen-related disorders. These disorders represent a group of rare conditions in which individuals may present with severe episodes of pathological bleeding and thrombosis; these conditions are treated by supplementing blood fibrinogen levels and inhibiting blood clotting, respectively. These disorders may also be the cause of certain liver and kidney diseases. Typical disorders may be selected from the group consisting of congenital afibrinogenemia, congenital hypofibrinogenemia, fibrinogen storage disease, congenital dysfibrinogenemia, hereditary fibrinogen A alpha chain amyloidosis, acquired dysfibrinogenemia, congenital hypodysfibrinogenemia, cryofibrinogenemia, and acquired hypofibrogenemia.

Moreover, the level of fibrinogen may increase in response to systemic inflammation, tissue injury, and certain other events including various cancers. Such elevated fibrinogen levels during inflammation as well as in cancer may be the cause of thrombosis and vascular injury accompanying these conditions.

The term “sample” as used herein refers to a liquid sample which comprises or is suspected to comprise fibrinogen. Said sample may be an artificial sample, such as solution comprising fibrinogen, or may be a naturally occurring sample, typically, a body fluid sample, more typically a blood sample or derivative thereof containing or suspected to contain fibrinogen, such as blood plasma or a fraction thereof. Typically, the blood sample is a whole blood sample or blood plasma sample.

The term “fibrinogen binding agent” as used herein refers to an agent comprising a first molecule which is capable of specifically binding fibrinogen and, reversibly bound to the said first molecule, a second molecule which is capable of specifically binding the first molecule. The affinity of said second molecule for the first molecule shall be lower than the affinity of fibrinogen for said first molecule.

Specific binding as used herein refers to the capability of the first molecule to bind fibrinogen but no other molecule present or suspected to be present in the sample. Thus, the first molecule shall have a high binding affinity for fibrinogen but a low or no binding affinity for other molecules except for the second molecule. However, as discussed elsewhere herein in detail, the binding affinity of the first molecule for the second molecule shall be lower than the binding affinity of the first molecule for fibrinogen. It is well known in the art how specificity of binding and/or affinity can be tested. Typically, affinity and/or specificity can be determined by using Surface Plasmon Resonance measurements of Radioisotope labeled ligand binding assays (see e.g., Vu 2013, Journal of Biological Chemistry, 288 (23): 16862-16871 or Stürzebecher 1986, Toxicon, 24 (6): 585-595).

First molecules having such binding properties for fibrinogen and the second molecule may be antibodies or aptamers.

An antibody which may be used as a first molecule in accordance with the present disclosure encompasses all types of antibodies that specifically bind to fibrinogen. Typically, the antibody used in the present methods is a monoclonal antibody, a polyclonal antibody, a single chain antibody, a chimeric antibody or any fragment or derivative of such antibodies being still capable of binding to fibrinogen specifically. Such fragments and derivatives comprised by the term antibody as used herein encompass a bispecific antibody, a synthetic antibody, a Fab, F(ab)2 Fv or scFv fragment, or a chemically modified derivative of any of these antibodies. Specific binding as used in the context of the antibody of the present disclosure means that the antibody does not cross react with other components in the sample. Specific binding can be tested by various well-known techniques. Antibodies or fragments thereof, in general, can be obtained by using methods which are described, e.g., in Harlow and Lane “Antibodies, A Laboratory Manual”, CSH Press, Cold Spring Harbor, 1988. Monoclonal antibodies may be prepared by known techniques, e.g., the fusion of mouse myeloma cells to spleen cells derived from immunized mammals and, preferably, immunized mice. Typically, an immunogenic peptide is applied to a mammal. The said peptide is, typically, conjugated to a carrier protein, such as bovine serum albumin, thyroglobulin, and keyhole limpet hemocyanin (KLH). Depending on the host species, various adjuvants can be used to increase the immunological response. Such adjuvants encompass, usually, Freund's adjuvant, mineral gels, e.g., aluminum hydroxide, and surface-active substances, e.g., lysolecithin, pluronic polyols, polyanions, peptides, oil emulsions, keyhole limpet hemocyanin, and dinitrophenol. Monoclonal antibodies which specifically bind to fibrinogen can be subsequently prepared using the well-known hybridoma technique, the human B cell hybridoma technique, and the EBV hybridoma technique.

An aptamer which can be used as a first molecule in accordance with the present disclosure encompasses oligonucleic acid or peptide molecules that bind to a specific analyte (Ellington 1990, Nature 346 (6287): 818-22; Bock 1992, Nature 355 (6360): 564-6). Oligonucleic acid aptamers are engineered through repeated rounds of selection or the so-called systematic evolution of ligands by exponential enrichment (SELEX technology). Peptide aptamers comprise of a variable peptide loop attached at both ends to a protein scaffold. This double structural constraint shall increase the binding affinity of the peptide aptamer into the nano-molar range. Said variable peptide loop length is, typically, composed of ten to twenty amino acids, and the scaffold may be any protein having improved solubility and compacity properties, such as thioredoxin-A. Peptide aptamer selection can be made using different systems including, e.g., the yeast two-hybrid system (see e.g., Hoppe-Seyler 2000, J Mol Med. 78 (8): 426-30).

The first molecule may also typically be an enzyme. The enzyme to be used in accordance with the present disclosure shall, typically, upon specific binding to said fibrinogen (i) not release fibrinogen or (ii) not cleave fibrinogen and release fibrinogen cleavage products. Thus, the enzyme may either be an enzyme which specifically binds to fibrinogen but is naturally not capable of cleaving it and thereby releasing the cleavage products. However, the term also encompasses enzymes which have been genetically modified to be capable of specifically binding fibrinogen but not cleaving it and releasing the cleavage products. Typically, such enzymes are inactivated in their active center by at least one amino acid exchange, deletion and/or addition. Also typically, such enzymes may be fragments of the whole enzyme comprising merely the substrate binding domain, i.e. the fibrinogen binding domain. Moreover, some enzymes require co-factors in order to cleave a substrate. If such enzymes specifically bind to fibrinogen, cleavage and release of the cleavage products can be prevented by omitting addition of the cofactor in the method of the present disclosure. More typically, said enzyme is a serine endopepdidase (E.C.3.4.21), preferably, a thrombin-like serine protease, more preferably, a thrombin-like serin protease having venombin A activity, most preferably, a batroxobin. More typically, batroxobin is batroxobin as described by You et al. (You 2004, FEBS Letters 571:67-73) and, in particular, batroxobin comprising an amino acid sequence as described in GenBank accession number J02684.1 or a variant thereof having venombin A activity. A variant of batroxobin as referred to in accordance with the present disclosure shall have an amino acid sequence which differs by at least one amino acid exchange, deletion and/or addition compared to the aforementioned reference sequence. However, it will be understood that the variant of batroxobin shall still have the binding properties of the first molecule referred to above.

The term “second molecule” as used in accordance with the present disclosure refers to a molecule which reversibly binds to the first molecule but has a lower affinity for the first molecule than fibrinogen. Typically, the affinity for fibrinogen is within the range of about K_{d} 0.5 μM to about 0.6 μM while the affinity for the second molecule, such as batroxobin, shall be above about K_{d} 0.6 μM. Care should be taken, however, that the first molecule in the initial fibrinogen binding agent still binds the second molecule with sufficient affinity such that the fibrinogen binding sites of the first molecule are all bound by second molecules.

Typically, the second molecule may comprise a tripeptide capable of specifically binding to the fibrinogen binding site of the first molecule with a lower binding affinity than fibrinogen. Typically, such a tripeptide shall have the following general formula:

N-terminus-X₁-X₂-X₃-C-terminus,

wherein X₃ is arginine or lysine. More typically, X₁ and/or X₂ are hydrophobic amino acids.

More typically, a tripeptide which is typically useful for a second molecule in accordance with the present disclosure is selected from the group consisting of:

N-terminus-Ile-Pro-Arg-C-terminus,

N-terminus-Tos-Gly-Pro-Arg-C-terminus, and

N-terminus-Tos-Gly-Pro-Lys-C-terminus,

wherein Tos refers to a tosyl moiety.

Yet, the second molecule in accordance with the present disclosure may also be a small molecule capable of specifically binding to the fibrinogen binding site of the first molecule with a lower binding affinity than fibrinogen. Typically, said small molecule is selected from the group consisting of N-alpha-substituted lysines, such as N-alpha acetyl-lysine-4-nitroanilide (CAS No. 50931-35-6), 4-amidinophenyl-alpha-aminobutyric acid amides, and 3- or 4-substituted benzamidines.

The second molecule according to the present disclosure shall comprises a detectable label and, typically, an optically or electrochemically detectable label.

An optically detectable label useful in accordance with the present disclosure may be typically selected from the group consisting of: a fluorophore which is inactive if the second molecule is bound to the first molecule, a chromophore which elicits fluorescence resonance energy transfer (FRET) if bound to the first molecule, and a chromogen which is inactive if the second molecule is bound to the first molecule.

An electrochemically detectable label useful in accordance with the present disclosure may be typically selected from the group consisting of: a ferrocene, PG 355 B, an osmium complex, a phenyldiamine, a nitrosoanilinderivate, and electrozyme.

Moreover, the electrochemical label may also be applied using signal enhancing oxidoreductase systems as described in EP1261861 B1 or EP0441222 B1.

Also typically, the said second molecule comprises a label that can be enzymatically activated. Typically, said label is an optically detectable label or an electrochemically detectable label as specified elsewhere herein. The second molecule as referred to herein may be enzymatically activated in that a label is released from the second molecule by enzymatic cleavage. Enzymes that can be used for cleavage of the second molecule depend on the nature of the second molecule. For example, if a tripeptide is applied as a second molecule in the method of the present disclosure, a peptidase may be used which cleaves the tripeptide such that a moiety comprising the detectable label is released which upon cleavage can be detected, i.e. upon cleavage a detectable signal will be generated. Enzymes which may be used for cleavage of the second molecule comprise peptidases, such as, thrombin, trypsin or plasmin. Furthermore, systems of proteases, as described in EP0018002 B1 may be applied.

Typically, if the second molecule comprises a tripeptide, the detectable label is linked to the C-terminus. However, the detectable label may also be linked to other amino acids of the tripeptide. It is to be understood, however, that the detectable label shall not interfere with binding of the tripeptide structure of the second molecule to the first molecule.

Depending on the detectable label, different detection techniques may be applied in order to determine the second molecule released from the first molecule.

Optically detectable labels may be determined by measuring light emission, fluorescence, FRET, polarization, refraction and the like. Typical optical detectors may be photomultipliers, phototubes, ionization detectors, active-pixel sensors, phototransistors, photodiodes, quantum dot photoconductors or photodiodes, photovoltaic cells, semiconductor detectors, thermal detectors, photochemical detectors and the like.

Electrochemically detectable labels may be determined by electrode arrangements such as thin layer electrodes suitable for voltametric and, typically, amperometric measurements. The detector, typically, contains at least two electrodes of which at least one electrode is a so-called working electrode. The electrodes may be composed of all conventional electrode materials such as metals, noble metals, alloys or graphite and are preferably composed of noble metals such as gold or palladium, or graphite. The various electrodes of the sensor can be composed of the same or different materials. More typically, the electrodes are made of palladium.

In view of the description herein, the second molecule may in particular be S-2288 (Isoleucyl-L-prolyl-L-arginine-p-nitroaniline), B7632 (N-Benzoyl-L-phenylalanyl-L-valyl-L-arginin-4-nitro-anilid, CAS No. 54799-93-8, CTHLys (Tos-Gly-Pro-Lys-pNA, CAS No. 73392-19-5), or LyspNA (Ac-Lys-pNA, CAS NO. 50931-35-6).

It will be understood that depending on the detectable label other detection techniques may also be suitable such as immunoassay-based detection techniques, mass determination, nucleic acid detection techniques and the like.

In the method of the present disclosure, a fibrinogen binding agent is provided. Said fibrinogen binding agent is a complex of two molecules, i.e. a first molecule and a second molecule, reversibly bound to each other. The first molecule has a binding site for fibrinogen and for the second molecule. The second molecule is capable of specifically binding the first molecule, wherein the affinity of said second molecule for the first molecule is lower than the affinity of fibrinogen for said first molecule. The fibrinogen binding agent can be provided, e.g., by incubating first molecules with an excess of the second molecule such that the fibrinogen binding sites of the first molecule are all bound by second molecules. Excessive second molecules can subsequently be removed, e.g., by washing steps. Typically, the first molecule may be immobilized on a suitable carrier, e.g., a carrier which can subsequently be used for or integrated in a detection device such as a test strip or an electrode.

The fibrinogen binding agent is brought into contact with a sample comprising or suspected to comprise fibrinogen, e.g., whole blood or blood plasma. The fibrinogen binding agent is contacted with the sample for a time and under conditions which allow for specific binding of fibrinogen to said first molecule. When fibrinogen binds to the first molecule, the second molecule will be released from the first molecule and, thus, from the fibrinogen binding agent. Thus, the second molecule is exchanged by fibrinogen due to the higher affinity of fibrinogen for the first molecule and as a result of this exchange, a new complex is formed comprising the first molecule and bound thereto fibrinogen.

The released second molecule in a further step can be determined by appropriate techniques as specified elsewhere herein in detail. For example, if the second molecule comprises or is linked to a detectable label, this detectable label can be determined. Suitable labels encompass chromogenic or fluorescent moieties which can be optically detected. It will be understood that based on the stoichiometry of the exchange, typically a ratio of 1:1, it can be assumed that a certain amount of fibrinogen present in the sample will release a certain amount of second molecules. Thus, determining the second molecules exchanged by fibrinogen and released from the first molecule allows for directly determining the fibrinogen present in the sample. For example, a calibration curve for the detectable signal elicited by the detectable label linked to the second molecule can be established using calibration samples comprising known amounts of fibrinogen. Subsequently, a measurement made in a test sample may be compared to the calibration curve and the amount of fibrinogen present in the test sample can be deduced from the calibration curve.

A typical method according to the disclosure is also described in the accompanying Examples, below.

Advantageously, it has been found in the studies underlying the present disclosure that using a preformed complex of a first molecule and a second molecule which can be detected as a fibrinogen binding agent can be effectively applied in a fibrinogen test. The second molecule shall have an affinity for the first molecule which is lower than the affinity of fibrinogen for the first molecule. Thereby, competition and as a consequence a release of the second molecule from the first molecule occurs if the fibrinogen binding agent is contacted with a sample comprising fibrinogen. Due to the stoichiometry of the exchange, there is a proportional relationship between the released second molecule and the fibrinogen bound to the first molecule which in turn reflects the fibrinogen originally present in the sample. Thus, the method of the present disclosure avoids the drawbacks of prior art methods which are based on reverse proportions between fibrinogen and detectable signals in particular under circumstances of low fibrinogen. Thanks to the present disclosure, a reliable determination of fibrinogen is possible over a wide range of concentrations. Moreover, the method is suitable for implementation in point-of-care diagnostic devices and can be easily automated.

The present disclosure also relates to a method for assessing coagulation defects or disorders in a subject comprising the steps of:
(aa) determining fibrinogen in a sample of said subject according to the aforementioned method of the disclosure for determining fibrinogen;
(bb) comparing the determined fibrinogen to a reference; and
(cc) assessing coagulation defects or disorders in said subject based on said comparison.

The term “assessing” as used herein refers to assessing whether a subject suffers from coagulation defects or disorders, or not, or whether said coagulation defects or disorders worsens or improves over time, or not. Accordingly, assessing as used herein includes identifying coagulation defects or disorders, identifying improvement or worsening of the said coagulation defects or disorders, monitoring coagulation defects or disorders, determining efficacy of a therapy of coagulation defects or disorders, and/or diagnosing coagulation defects or disorders. As will be understood by those skilled in the art, such an assessment, although preferred to be, may usually not be correct for 100% of the tested subjects. The term, however, requires that a statistically significant portion of subjects can be correctly assessed. Whether a portion is statistically significant can be determined without further ado by the person skilled in the art using various well known statistic evaluation tools, e.g., determination of confidence intervals, p-value determination, Student's t-test, Mann-Whitney test, etc. Details may be found in Dowdy and Wearden, Statistics for Research, John Wiley & Sons, New York 1983. Typically envisaged confidence intervals are at least 50%, at least 60%, at least 70%, at least 80%, at least 90%, at least 95%. The p-values are, typically, 0.2, 0.1, 0.05.

The term “coagulation defects or disorders” refers to any impairment of the physiologically coagulation cascade in a subject. Typically, said impairment will acquired or inherited. More typically, said coagulation defects or disorders are selected from the group consisting of: congenital afibrinogenemia, congenital hypofibrinogenemia, fibrinogen storage disease, congenital dysfibrinogenemia, hereditary fibrinogen A alpha chain amyloidosis, acquired dysfibrinogenemia, congenital hypodysfibrinogenemia, cryofibrinogenemia, and acquired hypofibrogenemia.

The term “comparing” as used herein encompasses comparing the amount of fibrinogen comprised by the sample to be analyzed with an amount of a suitable reference source specified elsewhere in this description. It is to be understood that comparing as used herein refers to a comparison of corresponding parameters or values, e.g., an absolute amount is compared to an absolute reference amount while a concentration is compared to a reference concentration. The comparison referred to in step (bb) of the method of the present disclosure may be carried out manually or computer assisted. For a computer assisted comparison, the value of the determined amount may be compared to values corresponding to suitable references which are stored in a database by a computer program. The computer program may further evaluate the result of the comparison, i.e. automatically provide the desired assessment in a suitable output format. Based on the comparison of the amount determined in step (aa) and the reference amount, it is possible to assess the coagulation defects or disorders. Therefore, the reference amount is to be chosen so that either a difference or a similarity in the compared amounts allows identifying those test subjects which belong into the group of subjects exhibiting coagulation defects or disorders. The method allows either excluding (rule-out) or identifying (rule-in) subject as a subject suffering from coagulation defects or disorders.

The term “reference amount” as used herein refers to an amount which allows for allocation of a subject into either the group of subjects coagulation defects or disorders or the group of healthy subjects with respect to coagulation defects or disorders. Such a reference amount can be a threshold amount which separates these groups from each other. Accordingly, the reference amount shall be an amount which allows for allocation of a subject into a group of subjects exhibiting coagulation defects or disorders, or not. A suitable threshold amount separating the two groups can be calculated without further ado by the statistical tests referred to herein elsewhere based on amounts of fibrinogen from either a subject or group of subjects known to suffer from coagulation defects or disorders or a subject or group of subjects known not to suffer from coagulation defects or disorders. Typically, if said reference amount is derived from a subject or a group of subjects known to suffer from coagulation defects or disorders, an essentially identical or decreased amount of fibrinogen is indicative for a subject having coagulation defects or disorders. Also typically, if said reference amount is derived from a subject or a group of subjects known not to suffer from coagulation defects or disorders, an essentially identical or increased amount of fibrinogen is indicative for a subject not suffering from coagulation defects or disorders. The reference amount applicable for an individual subject may vary depending on various physiological parameters such as age, gender, or subpopulation.

Reference amounts can, in principle, be calculated for a cohort of subjects based on the average or mean values for a given parameter such as fibrinogen amount by applying standard statistically methods. In particular, accuracy of a test such as a method aiming to diagnose an event, or not, is best described by its receiver-operating characteristics (ROC) (see especially Zweig 1993, Clin. Chem. 39:561-577). The ROC graph is a plot of all of the sensitivity/specificity pairs resulting from continuously varying the decision threshold over the entire range of data observed. The clinical performance of a diagnostic method depends on its accuracy, i.e. its ability to correctly allocate subjects to a certain prognosis or diagnosis. The ROC plot indicates the overlap between the two distributions by plotting the sensitivity versus 1-specificity for the complete range of thresholds suitable for making a distinction. On the y-axis is sensitivity, or the true-positive fraction, which is defined as the ratio of number of true-positive test results to the product of number of true-positive and number of false-negative test results. This has also been referred to as positivity in the presence of a disease or condition. It is calculated solely from the affected subgroup. On the x-axis is the false-positive fraction, or 1-specificity, which is defined as the ratio of number of false-positive results to the product of number of true-negative and number of false-positive results. It is an index of specificity and is calculated entirely from the unaffected subgroup. Because the true- and false-positive fractions are calculated entirely separately, by using the test results from two different subgroups, the ROC plot is independent of the prevalence of the event in the cohort. Each point on the ROC plot represents a sensitivity/-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. If the ROC plot falls completely below the 45° diagonal, this is easily remedied by reversing the criterion for “positivity” from “greater than” to “less than” or vice versa. Qualitatively, the closer the plot is to the upper left corner, the higher the overall accuracy of the test. Dependent on a desired confidence interval, a threshold can be derived from the ROC curve allowing for the diagnosis or prediction for a given event with a proper balance of sensitivity and specificity, respectively. Accordingly, the reference to be used for the aforementioned method of the present disclosure, i.e. a threshold which allows to discriminate between subjects suffering from coagulation defects and those who do not suffer therefrom can be generated, usually, may be selected by establishing a ROC for said cohort as described above and deriving a threshold amount therefrom. Dependent on a desired sensitivity and specificity for a diagnostic method, the ROC plot allows deriving suitable thresholds. It will be understood that an optimal sensitivity is desired for excluding a subject for being at increased risk (i.e. a rule out) whereas an optimal specificity is envisaged for a subject to be assessed as being at an increased risk (i.e. a rule in).

Typically, the physiological level of fibrinogen may be used as reference. More typically, said physiological range is between about 1.5 to 3.0 mg/ml.

The present disclosure also relates to a device for determining fibrinogen in a sample comprising: (A) an analyzing unit comprising (i) a reaction zone comprising a fibrinogen binding agent, said fibrinogen binding agent comprising a first molecule which is capable of specifically binding fibrinogen and, reversibly bound to the said first molecule, a second molecule which is capable of specifically binding the first molecule, wherein the affinity of said second molecule for the first molecule is lower than the affinity of fibrinogen for said first molecule, wherein said reaction zone is configured for contacting the fibrinogen binding agent with a sample suspected to comprise fibrinogen for a time and under conditions which allow for specific binding of fibrinogen to said first molecule whereby the second molecule is released from the first molecule and fibrinogen is specifically bound by the first molecule; and (ii) a detector which is capable of determining the second molecule released from the fibrinogen binding agent; and (B)) an evaluation unit which is capable of determining fibrinogen in the sample based on the released second molecule.

The term “device” as used herein relates to a system comprising the aforementioned units operatively linked to each other as to allow the determination of fibrinogen according to the methods of the present disclosure.

The analyzing unit, typically, comprises a reaction zone having said fibrinogen binding agent in immobilized form on a solid support or carrier which is to be contacted to the sample comprising the fibrinogen or suspect to comprise fibrinogen. Moreover, in the reaction zone, it is possible to apply conditions which allow for the exchange of the second molecule of the fibrinogen binding agent by fibrinogen from the sample.

The reaction zone may either allow directly for sample application or it may be connected to a loading zone where the sample is applied. In the latter case, the sample can be actively or passively transported via the connection between the loading zone and the reaction zone to the reaction zone. Moreover, the reaction zone shall be also connected to a detector. The connection shall be such that the detector can detect the second molecules released from the first molecules of the fibrinogen binding agents. Suitable connections depend on the techniques used for measuring the presence or amount of the second molecules. For example, for optical detection, transmission of light may be required between the detector and the reaction zone while for electrochemical determination a fluidal connection may be required, e.g., between the reaction zone and an electrode.

The detector may be adapted to detect determine the amount of second molecules which are released. The determined amount can be subsequently transmitted to the evaluation unit. Said evaluation unit comprises a data processing element, such as a computer, with an implemented algorithm for determining the amount of fibrinogen present in the sample based on the measured amount of second molecules.

The present disclosure, further, relates to a kit for determining fibrinogen in a sample comprising a fibrinogen binding agent, said fibrinogen binding agent comprising a first molecule which is capable of specifically binding fibrinogen and, reversibly bound to the said first molecule, a second molecule which is capable of specifically binding the first molecule, wherein the affinity of said second molecule for the first molecule is lower than the affinity of fibrinogen for said first molecule.

The term “kit” as used herein refers to a collection of the aforementioned components, typically, provided in separately or within a single container. The container also typically comprises instructions for carrying out the methods of the present disclosure. These instructions may be in the form of a manual or may be provided by a computer program code which is capable of carrying out or supports the determination of fibrinogen referred to in the methods of the present disclosure when implemented on a computer or a data processing device. The computer program code may be provided on a data storage medium or device such as an optical storage medium (e.g., a Compact Disc) or directly on a computer or data processing device or may be provided in a download format such as a link to an accessible server or cloud. Moreover, the kit may, usually, comprise standards for reference amounts of fibrinogen for calibration purposes as described elsewhere herein in detail. The kit according to the present disclosure may also comprise further components necessary for carrying out the methods described herein, such as solvents, washing solutions and/or reagents required for detection of the released second molecule. Further, it may comprise any of the devices described herein, either in parts or in their entirety.

The aforementioned devices and kits may be for use in assessing coagulation defects or disorders in a subject.

The present disclosure, in general, also relates to a fibrinogen binding agent, said fibrinogen binding agent comprising a first molecule which is capable of specifically binding fibrinogen and, reversibly bound to the said first molecule, a second molecule which is capable of specifically binding the first molecule, wherein the affinity of said second molecule for the first molecule is lower than the affinity of fibrinogen for said first molecule. Further contemplated is the use of said fibrinogen binding agent for determining fibrinogen in a sample

It is to be understood that the definitions and explanations of the terms made above apply accordingly for all embodiments described in this specification and the accompanying claims. The following embodiments are particular embodiments envisaged according to the present disclosure:

Summarizing and without excluding further possible embodiments, the following embodiments may be envisaged:

Embodiment 1: A method for determining fibrinogen in a sample comprising the steps of:
(a) contacting a fibrinogen binding agent,
   said fibrinogen binding agent comprising a first molecule which is capable of specifically binding fibrinogen and, reversibly bound to the said first molecule, a second molecule which is capable of specifically binding the first molecule, wherein the affinity of said second molecule for the first molecule is lower than the affinity of fibrinogen for said first molecule,
   with a sample suspected to comprise fibrinogen for a time and under conditions which allow for specific binding of fibrinogen to said first molecule, whereby the second molecule is released from said first molecule and fibrinogen is specifically bound by the first molecule;
(b) determining said second molecule released from said first molecule; and
(c) determining fibrinogen in the sample based on the released second molecule.

Embodiment 2: The method of Embodiment 1, wherein said sample is a body fluid sample, preferably, a blood plasma sample or a whole blood sample.

Embodiment 3: The method of Embodiment 1 or 2, wherein said first molecule is an antibody or a aptamer.

Embodiment 4: The method of Embodiment 1 or 2, wherein said first molecule is an enzyme.

Embodiment 5: The method of Embodiment 4, wherein said enzyme upon specific binding to said fibrinogen (i) does not release fibrinogen or (ii) does not cleave fibrinogen and release fibrinogen cleavage product.

Embodiment 6: The method of Embodiment 4 or 5, wherein said enzyme is a serine endopeptidase (E.C.3.4.21), preferably, a thrombin-like serine protease, more preferably, a thrombin-like serine protease having venombin A activity, most preferably, a batroxobin.

Embodiment 7: The method of any one of Embodiments 1 to 6, wherein said second molecule comprises a tripeptide capable of specifically binding to the fibrinogen-binding site of the first molecule.

Embodiment 8: The method of Embodiment 7, wherein said tripeptide has the following general formula: N-terminus-X₁-X₂-X₃-C-terminus, wherein X₃ is arginine or lysine.

Embodiment 9: The method of Embodiment 8, wherein X₁ and/or X₂ are hydrophobic amino acids.

Embodiment 10: The method of Embodiment 7, wherein the tripeptide is selected from the group consisting of N-terminus-Ile-Pro-Arg-C-terminus, N-terminus-Tos-Gly-Pro-Arg-C-terminus, N-terminus-Pro-Phe-Arg-C-terminus, and N-terminus-Tos-Gly-Pro-Lys-C-terminus.

Embodiment 11: The method of any one of Embodiments 1 to 6, wherein said second molecule is selected from the group consisting of N-alpha-substituted lysines, in an embodiment N-alpha acetyl-lysine-4-nitroanilide (CAS No. 50931-35-6), 4-amidinophenyl-alpha-aminobutyric acid amides, modified aminoiminomethylamino piperidinecarboxylic acids with bulky hydrophobic sidechains, modified argatroban, and 3- or 4-substituted benzamidines.

Embodiment 12: The method of any one of Embodiments 1 to 11, wherein said second molecule comprises a detectable label.

Embodiment 13: The method of Embodiment 12, wherein said label is an optically detectable label.

Embodiment 14: The method of Embodiment 13, wherein said optically detectable label is selected from the group consisting of: a fluorophore which is inactive if the second molecule is bound to the first molecule, a chromophore which elicits FRET if bound to the first molecule, and a chromogen which is inactive if the second molecule is bound to the first molecule.

Embodiment 15: The method of Embodiment 14, wherein said label is an electrochemically detectable label.

Embodiment 16: The method of Embodiment 15, wherein said electrochemically detectable label is selected from the group consisting of: a ferrocene, PG 355 B, an osmium complex, a phenyldiamin, a nitrosoanilin derivative, and electrozyme.

Embodiment 17: The method of any one of Embodiments 1 to 11, wherein said second molecule comprises a label that can be enzymatically activated.

Embodiment 18: The method of Embodiment 17, wherein said label is an optically detectable label or a electrochemically detectable label.

Embodiment 19: The method of Embodiments 12 to 16 wherein the second molecule comprises a tripeptide as specified in any one of embodiments 7 to 10 and wherein said label is linked to the C-terminus of the tripeptide.

Embodiment 20: A method for assessing coagulation defects or disorders in a subject comprising the steps of:
(aa) determining fibrinogen in a sample of said subject according to the method of any one of embodiments 1 to 18;
(bb) comparing the determined fibrinogen to a reference; and
(cc) assessing coagulation defects or disorders in said subject based on said comparison.

Embodiment 21: The method of Embodiment 19, wherein said coagulation defects or disorders are selected from the group consisting of: congenital afibrinogenemia, congenital hypofibrinogenemia, fibrinogen storage disease, congenital dysfibrinogenemia, hereditary fibrinogen A alpha chain amyloidosis, acquired dysfibrinogenemia, congenital hypodysfibrinogenemia, cryofibrinogenemia, and acquired hypofibrogenemia.

Embodiment 22: A device for determining fibrinogen in a sample comprising:
(A) an analyzing unit comprising
   (i) a reaction zone comprising a fibrinogen binding agent, said fibrinogen binding agent comprising a first molecule which is capable of specifically binding fibrinogen and, reversibly bound to the said first molecule, a second molecule which is capable of specifically binding the first molecule, wherein the affinity of said second molecule for the first molecule is lower than the affinity of fibrinogen for said first molecule, wherein said reaction zone is configured for contacting the fibrinogen binding agent with a sample suspected to comprise fibrinogen for a time and under conditions which allow for specific binding of fibrinogen to said first molecule whereby the second molecule is released from the first molecule and fibrinogen is specifically bound by the first molecule; and
   (ii) a detector which is capable of determining the second molecule released from the fibrinogen binding agent;
   and
(B) an evaluation unit which is capable of determining fibrinogen in the sample based on the released second molecule.

Embodiment 23: A kit for determining fibrinogen in a sample comprising a fibrinogen binding agent, said fibrinogen binding agent comprising a first molecule which is capable of specifically binding fibrinogen and, reversibly bound to the said first molecule, a second molecule which is capable of specifically binding the first molecule, wherein the affinity of said second molecule for the first molecule is lower than the affinity of fibrinogen for said first molecule.

Embodiment 24: A kit according to Embodiment 23 for use in determining assessing coagulation defects or disorders in a subject.

Embodiment 25: Use of a fibrinogen binding agent, said fibrinogen binding agent comprising a first molecule which is capable of specifically binding fibrinogen and, reversibly bound to the said first molecule, a second molecule which is capable of specifically binding the first molecule, wherein the affinity of said second molecule for the first molecule is lower than the affinity of fibrinogen for said first molecule for use in determining fibrinogen in a sample.

Embodiment 26: A fibrinogen binding agent, said fibrinogen binding agent comprising a first molecule which is capable of specifically binding fibrinogen and, reversibly bound to the said first molecule, a second molecule which is capable of specifically binding the first molecule, wherein the affinity of said second molecule for the first molecule is lower than the affinity of fibrinogen for said first molecule for use in determining assessing coagulation defects or disorders in a subject.

Embodiment 27: A fibrinogen binding agent, said fibrinogen binding agent comprising a first molecule which is capable of specifically binding fibrinogen and, reversibly bound to the said first molecule, a second molecule which is capable of specifically binding the first molecule, wherein the affinity of said second molecule for the first molecule is lower than the affinity of fibrinogen for said first molecule.

All patents, published applications, research papers, and any other references cited in this specification are herewith incorporated by reference with respect to their entire disclosure content and the disclosure content specifically mentioned in this specification.

### EXAMPLES

The following Examples shall merely illustrate aspects of the present disclosure, which is defined solely by the claims. These Examples shall not be construed whatsoever as limiting its scope.

### Example 1: Fibrinogen Competition Assay Using Batroxobin

An assay for determining fibrinogen in a sample was developed which allows to determine fibrinogen levels based on a direct proportional relationship rather than using inverse proportional relationships as is the case in the available competition assays, such as described in WO2019/068940.

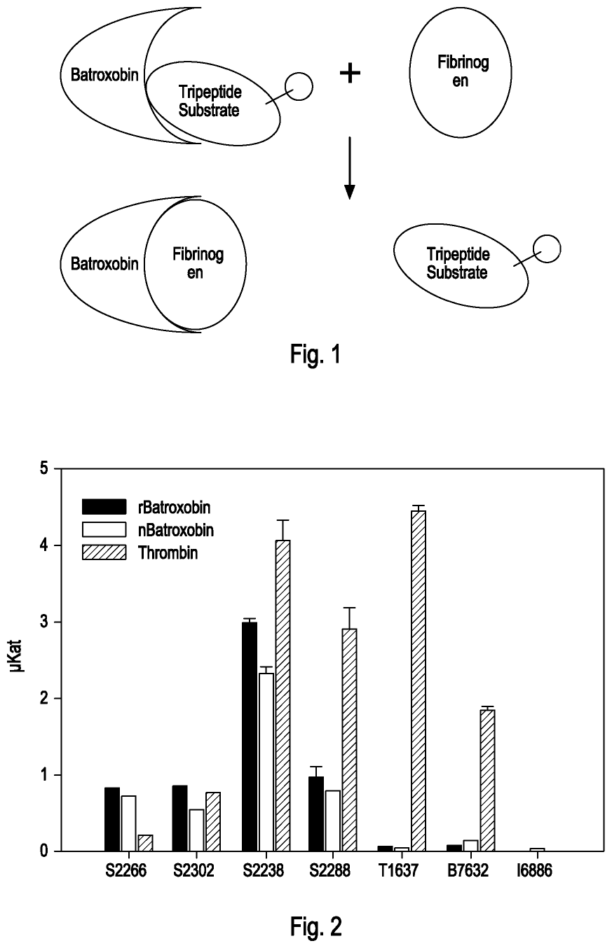

The principle of such an assay for measuring fibrinogen is shown in FIG. 1. In said assay, a complex of batroxobin and a substrate, usually, linked to a detectable label is applied. From said complexes, the substrate is released after the complex has been contacted with fibrinogen, e.g. present in a sample. The released substrate can exemplarily be determined afterwards by different techniques: (A) the tripeptide substrate may comprise a dye, e.g. a fluorophore, which has a reduced fluorescence when the tripeptide substrate is bound to Batroxobin (i.e. is quenched), thus, the more tripeptide substrate is released, the higher the detectable fluorescence signal; (B) the tripeptide substrate may comprise a redox-active group, e.g. a group which can be oxidized at an appropriate electrode. Thus, the more tripeptide substrate is released, the higher the detectable electrochemical signal; (C) the tripeptide substrate may comprise a chromogenic group changing its spectral properties after cleavage from the tripeptide substrate; thus, the more tripeptide substrate is released, the higher e.g. a remission, absorption, or florescence signal is detected.

A prerequisite for a suitable substrate for such an assay is a specific range of the binding affinity to batroxobin while ensuring no or low cleavage of the substrate by batroxobin. The affinity needs to be high (and specific) enough to enable stoichiometric saturation of the available batroxobin binding pockets but lower than the binding affinity of fibrinogen to batroxobin to enable displacement of the substrate by fibrinogen. The binding affinity of batroxobin to both circulating fibrinogen isoforms γA-γA and γA/γ′-fibrinogen is mediated via a single high affinity site (Kd values of 0.6±0.1 μM and 0.50±0.04 μM, respectively, (Vu 2013, loc. cit.)). A suitable substrate should thus have a Kd value >0.6 μM while still enabling binding most of the substrate in the initial state of the reaction.

Parameters characterizing suitable serine protease substrates, which usually contain an arginine as the C-terminal amino acid, were identified. Arginine is recognized as cleavage site by both thrombin and batroxobin but binding preferences in the binding pocket for peptide substrates are slightly different. For detection variant C, where signal generation is achieved by addition of a second enzyme, e.g. thrombin, further cleavage by the second enzyme must be possible. Low-level cleavage by batroxobin with C-terminal arginine substrates on the other hand is required to ensure sufficient binding of the substrate to the batroxobin active site for detection variant A, B or C. Two chromogenic substrates suitable for this application are B7632 and S-2288 (see FIG. 2).

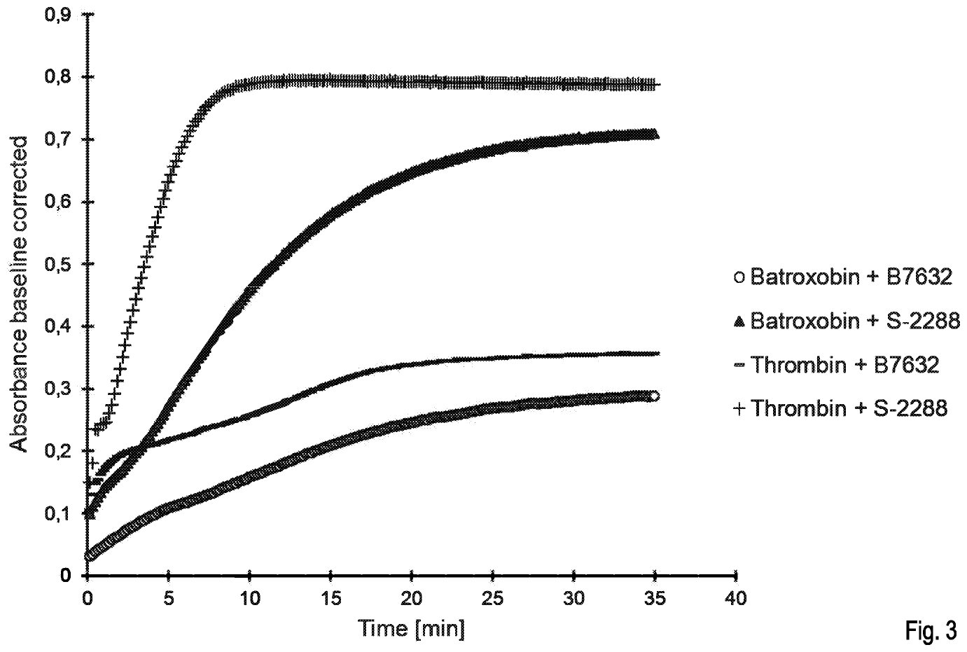

When monitoring cleavage of the substrate by either batroxobin or thrombin with both substrates and enzymes at optimal concentrations with a photometer until saturation, similar differences in absorbance could be reproduced (see FIG. 3).

When we compared the times needed to reach 50% of the maximal absorbance, we found that for B7632, the time to reach 50% absorbance for thrombin is approximately 15% of the time needed with batroxobin whereas it is approximately 40% for S-2288 (see Table 1).

TABLE 1
Comparison of times needed to reach 50% of the maximal absorbance
(t(50% absorbance)) for catalysis of B7632 or S-2288 by
batroxobin and thrombin, respectively. The last column indicates
the percentage of t(50% absorbance) for the respective substrate
for thrombin in relation to batroxobin.

T(50% absorbance) [min]

| Substrate | batroxobin | thrombin | T. % batroxobin |
|---|---|---|---|
| B7632 | 8.7 | 1.3 | 14.9 |
| S-2288 | 7 | 2.7 | 38.6 |

In addition, a sufficient cleavage of the substrate and a sigmoidal rise in chromogenic signal in response to the presence of thrombin is required for a suitable assay since inefficient cleavage by thrombin would add additional complexity to the 2-step reaction. In this respect, B7632 is usable, but quickly cleaved substrates like S-2288 may be preferred as substrate (see FIG. 3).

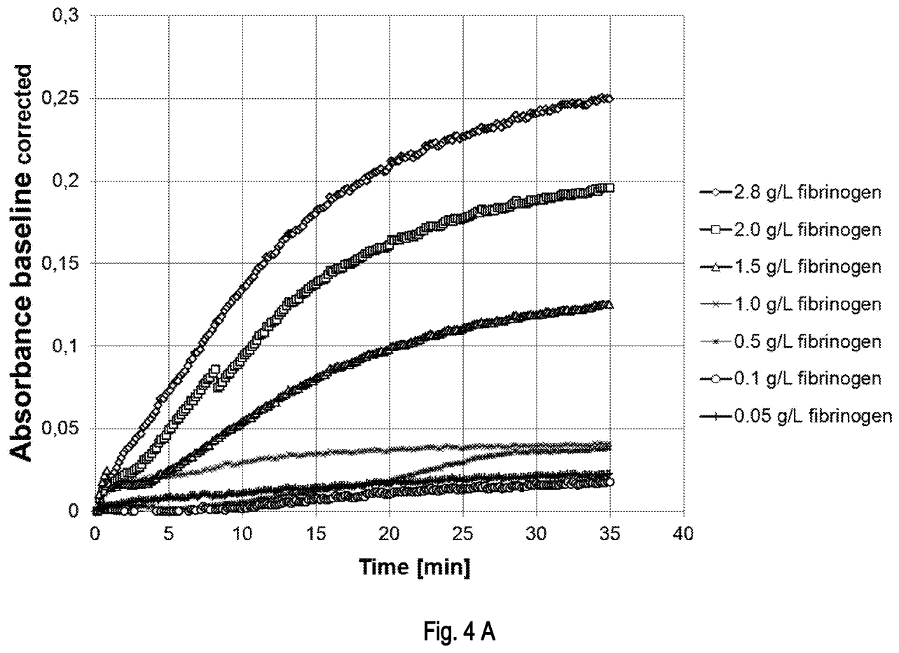

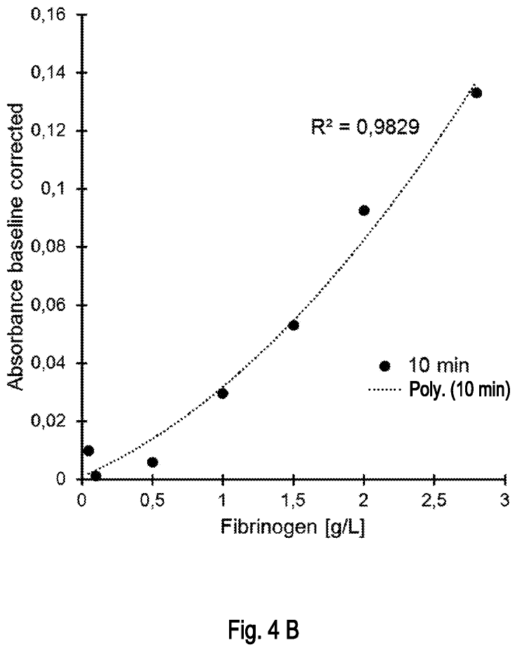

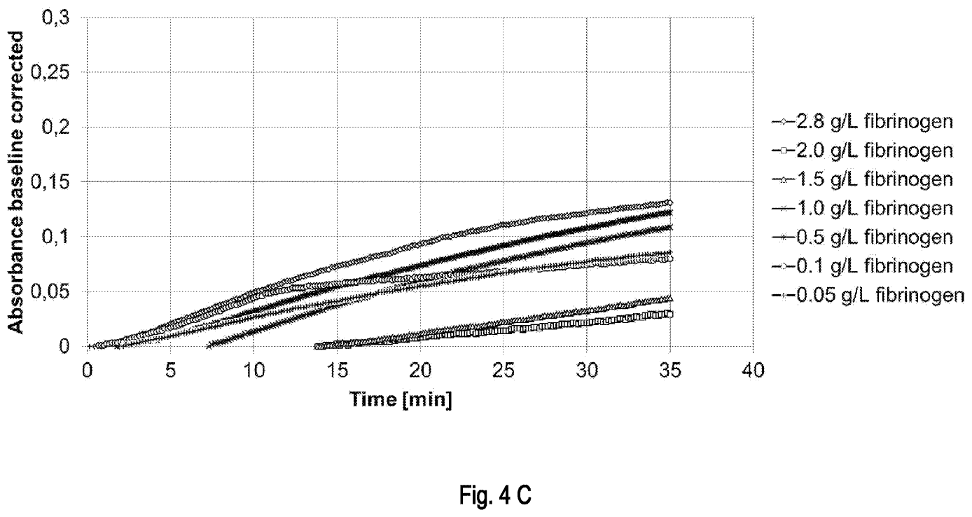

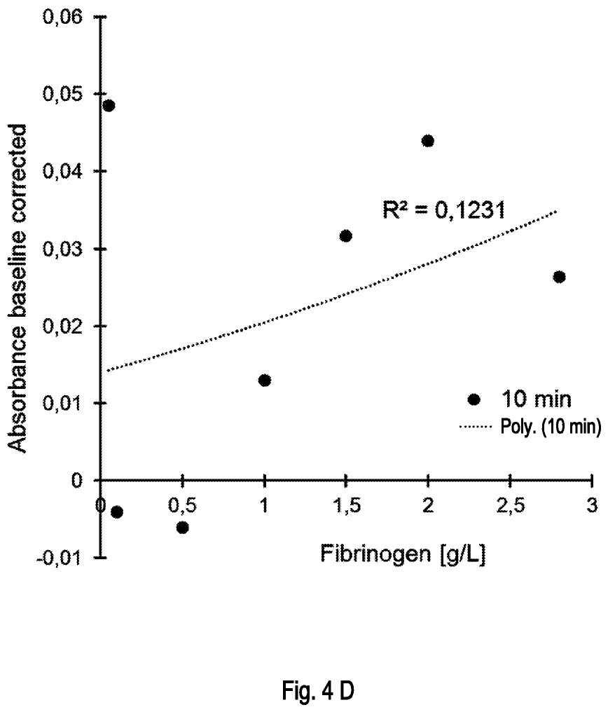

When performing the 2-step reaction with a pre-incubation of different plasma fibrinogen levels with the substrates (200 μM), batroxobin (7 U/mL) and the fibrin polymerization blocker pefabloc FG (2.3 mM) for 10 min at 37 °C and subsequent addition of thrombin (0.1 U/mL) while monitoring absorbance with a photometer, very good direct correlation with fibrinogen content for S-2288 but not for B7632 (see FIG. 4) was found. This direct correlation of the signal to fibrinogen content generated by the second enzyme is the final confirmation step needed to identify a suitable substrate. Alternatively to confirmation of the direct correlation of substrate release to fibrinogen content by a second enzyme (detection variant C), the suitability of a substrate can also be confirmed by direct optical or electrochemical signal generation with a respective chemical group (detection variants A and B).

Therefore, it is conclude that the prerequisite for a suitable substrate containing a C-terminal arginine is a ratio of time to half-maximal absorbance of <0.5 for thrombin in comparison to batroxobin under the described wet chemistry conditions and a sufficient cleavage rate with an absorbance >0.5 for thrombin under optimized assay conditions.

In principle, other substrates which show sufficient affinity to batroxobin with limited or no cleavage by batroxobin can also be employed in this assay. For example, the tripeptidic plasmin substrate CTHLys (Tos-Gly-Pro-Lys-pNA) is almost identical to the serine protease substrate chromozym TH (Tos-Gly-Pro-Arg-pNA) with a replacement of the C-terminal arginine by the chemically similar lysine. In this case, if affinity to batroxobin is high enough to enable initial binding of most of the substrate to batroxobin, plasmin would be used as a second enzyme instead of thrombin to elicit substrate cleavage and signal generation and represents an alternative approach for detection variant C, where signal generation is achieved by addition of a second enzyme. Both CTHLys (Tos-Gly-Pro-Lys-pNA, CAS No. 73392-19-5) and LyspNA (Ac-Lys-pNA, CAS NO. 50931-35-6) are known plasmin substrates. CTHLys is very efficiently cleaved by plasmin, LyspNA is also a plasmin substrate but is cleaved ca. 2.5× less efficiently.

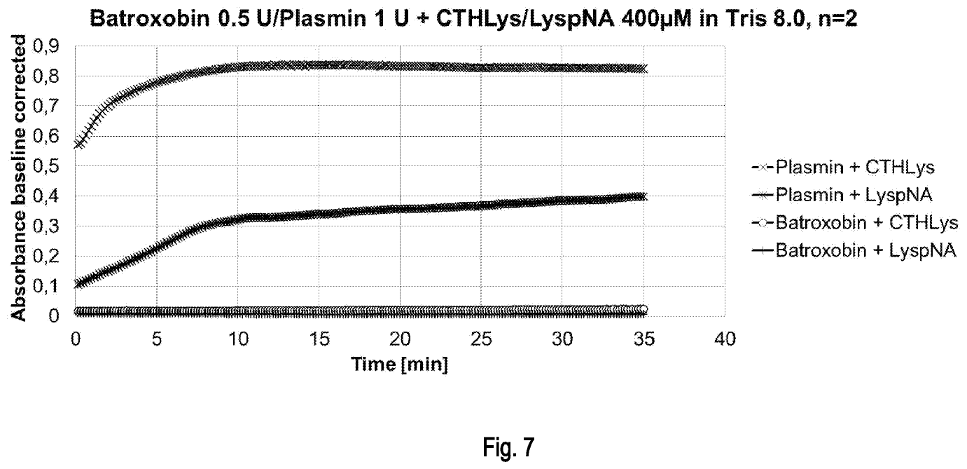

No cleavage by batroxobin (7 U/mL) of the two substrates CTHLys (400 μM) and LyspNA (400 μM) and efficient cleavage of CTHLys (400 μM) by plasmin and less efficient cleavage of LyspNA (400 μM) by plasmin (14 U/mL) were experimentally confirmed (FIG. 7). The offset of the starting points for plasmin samples was caused by a delay in measurement caused by pipetting speed.

Additional experiments with a 2-step reaction with a pre-incubation of different plasma fibrinogen levels with the substrates, batroxobin, the fibrin polymerization blocker pefabloc FG and subsequent addition of plasmin while monitoring absorbance with a photometer similar to the experiments with the thrombin substrates above would need to be conducted to establish good direct correlation with fibrinogen content for this approach.

Another possible option would be the use or modification of known specific inhibitors of batroxobin (Stürzebecher 1986, loc. cit.). A specific inhibitor should have high affinity to batroxobin. In addition, this inhibition needs to be reversible in a dose-dependent manner by the presence of fibrinogen. The detection could be accomplished by adding a fluorophore, a chromogenic or electrochemically active group.

### Example 2: Detection of Released Substrates

### Variant A—Signal Generation Via Fluorophore

In substrates which were identified to be suitable for the assay in wet chemistry testing, the functional chromogenic group (e.g. pNA in S-2288) can be replaced by a fluorophore, for example, 7-amino-4-methylcoumarine.

### Variant B—Electrochemical Signal Generation

### (1) Reduction of the Nitro Group in Nitrosoaniline Chromogenic Substrates

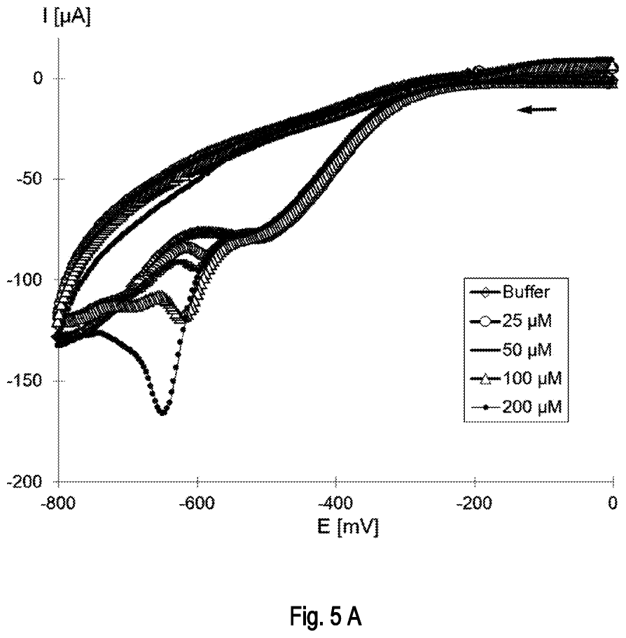

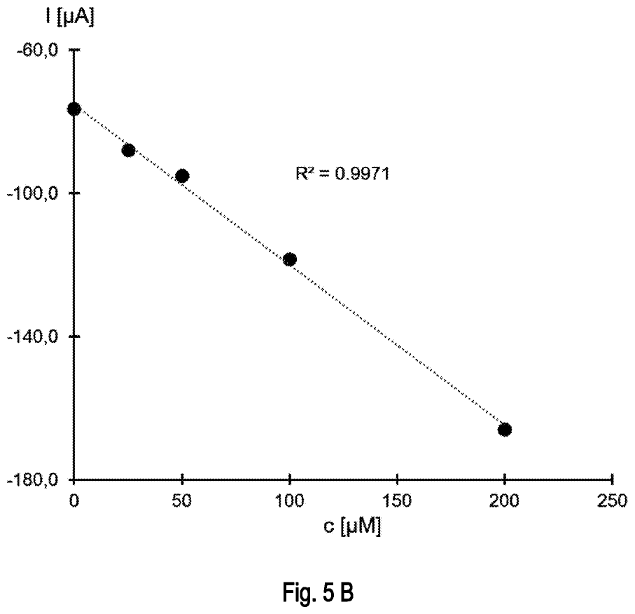

A proof-of-principle experiment showed that the nitro group of the substrate S-2288 can be reduced in the range of −500 to −800 mV in a specific, dose-dependent manner (see FIG. 5). This correlation can be used to monitor substrate released from binding to batroxobin by direct reduction at e.g. a test strip electrode.

### (2) Replacement of the Functional Chromogenic Group in Suitable Substrates

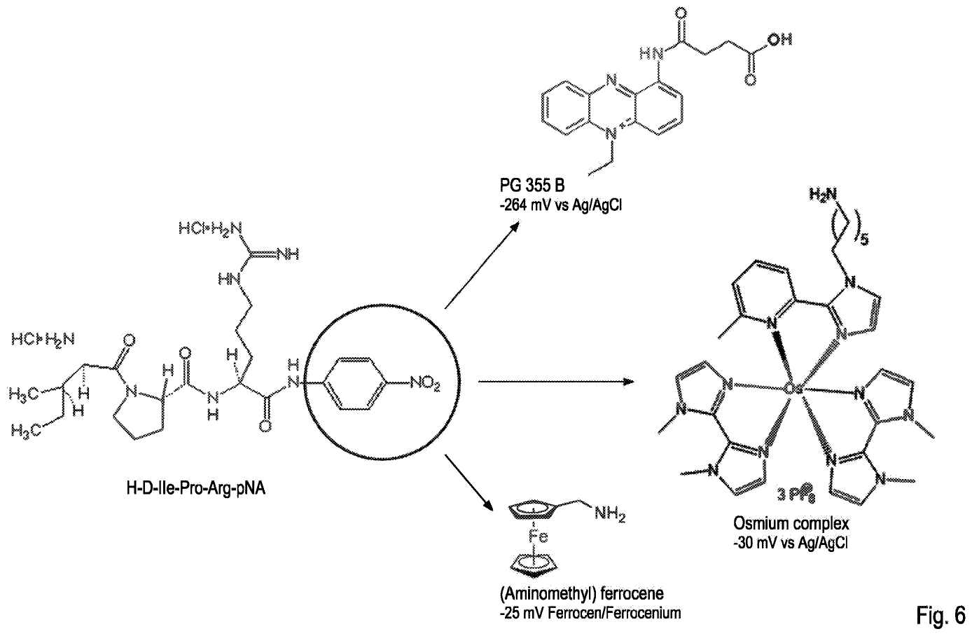

Chromogenic groups in suitable substrates can also be replaced by electrochemically active groups. To limit adverse effects on test performance due to interference of oxidation or reduction of other dry chemistry or sample components, a resting potential of the complex between −500 mV and +200 mV against Ag/AgCl may be used. Suitable electrochemically active groups are, e.g., PG 355 B, an osmium complex or ferrocene (see FIG. 6).

### Variant C—Substrate Cleavage by a Second Enzyme

For detection via a second enzyme, the release of the substrate from the binding to batroxobin by fibrinogen-induced displacement would be monitored by subsequent cleavage of the free substrate by a second enzyme. Suitable second enzymes need to be able to process the substrate efficiently. Serine protease substrates like S-2288 can processed with thrombin as mentioned above or other related proteases with sufficient activity. The activity of the second enzyme influences the total processing time of the assay so a high activity towards the respective substrate may be used. This requires a timed, sequential reaction with special and/or temporal separation of the consecutive enzyme reactions. Depending on the desired assay format, detection can e.g. be achieved by a fluorophore, a chromogenic, or an electrochemically active group attached to the substrate as alluded to earlier (detection variant A or B).

### Example 3: CTHLys or LyspNA as Substrates

To test the hypothesis that other substrates and secondary enzymes for Variant C can be employed, experiments with the tripeptidic plasmin substrate CTHLys (Tos-Gly-Pro-Lys-pNA) and LyspNA (Ac-Lys-pNA) were conducted. Plasmin would be used as a second enzyme instead of thrombin to elicit substrate cleavage and signal generation.

Both CTHLys (Tos-Gly-Pro-Lys-pNA, CAS No. 73392-19-5) and LyspNA (Ac-Lys-pNA, CAS NO. 50931-35-6) as substrates are not cleaved by batroxobin; CTHLys is very efficiently cleaved by plasmin, LyspNA is also a plasmin substrate but is cleaved ca. 2.5× less efficiently. The offset of the starting points for plasmin samples was caused by a delay in measurement caused by pipetting speed.

### LITERATURE CITED

1. Bock 1992, Nature 355 (6360): 564-6.
2. Ellington 1990, Nature 346 (6287): 818-22.
3. European Patent No. EP 0 018 002 B1.
4. European Patent No. EP 0 441 222 B1.
5. European Patent No. EP 1 261 861 B1.
6. Heimann 1979, J Clin Chem Clin Biochem 17 (6) 369-72.
7. Hoppe-Seyler 2000, J Mol Med. 78 (8): 426-30.
8. Mackie et al, Thromb Haemost. 2002 June; 87 (6): 997-1005.
9. Stürzebecher 1986, Toxicon, 24 (6): 585-595.
10. Vu 2013, Journal of Biological Chemistry, 288 (23): 16862-16871.
11. International Patent Application Pub. No. WO2019/068940A1.
12. You 2004, FEBS Letters 571:67-73.
13. Zweig 1993, Clin. Chem. 39:561-577.

1. A method for determining fibrinogen in a sample, comprising the steps of:
(a) contacting a fibrinogen binding agent,
said fibrinogen binding agent comprising a first molecule which is capable of specifically binding fibrinogen and, reversibly bound to the said first molecule, a second molecule which is capable of specifically binding the first molecule,
wherein the affinity of said second molecule for the first molecule is lower than the affinity of fibrinogen for said first molecule,
with a sample suspected to comprise fibrinogen for a time and under conditions which allow for specific binding of fibrinogen to said first molecule, whereby the second molecule is released from said first molecule and fibrinogen is specifically bound by the first molecule;
(b) determining said second molecule released from said first molecule; and
(c) determining fibrinogen in the sample based on the released second molecule.

2. The method of claim 1, wherein said sample is a body fluid sample, optionally, a blood plasma sample or a whole blood sample.

3. The method of claim 1, wherein said first molecule is an antibody or an aptamer.

4. The method of claim 1, wherein said first molecule is an enzyme.

5. The method of claim 4, wherein said enzyme upon specific binding to said fibrinogen (i) does not release fibrinogen or (ii) does not cleave fibrinogen and release fibrinogen cleavage product.

6. The method of claim 4, wherein said enzyme is a serine endopeptidase (E.C.3.4.21), optionally, a thrombin-like serine protease.

7. The method of claim 1, wherein said second molecule comprises a tripeptide capable of specifically binding to the fibrinogen binding site of the first molecule.

8. The method of claim 7, wherein the tripeptide is selected from the group consisting of N-terminus-Ile-Pro-Arg-C-terminus, N-terminus-Tos-Gly-Pro-Arg-N-terminus, N-terminus-Pro-Phe-Arg-C-terminus and N-terminus-Tos-Gly-Pro-Lys-C-terminus.

9. The method of claim 1, wherein said second molecule is selected from the group consisting of N-alpha-substituted lysines, 4-amidinophenyl-alpha-aminobutyric acid amides, modified aminoiminomethylamino piperidinecarboxylic acids with bulky hydrophobic sidechains, modified argatroban, and 3- or 4-substituted benzamidines.

10. The method of claim 1, wherein said second molecule comprises a detectable label.

11. The method of claim 10, wherein said detectable label is an optically detectable label or an electrochemically detectable label.

12. The method of claim 1, wherein said second molecule comprises a label that can be enzymatically activated.

13. The method of claim 6, wherein said enzyme is a thrombin-like serine protease having venombin A activity, optionally, a batroxobin.

14. The method of claim 9, wherein said second molecule is N-alpha acetyl-lysine-4-nitroanilide (CAS No. 50931-35-6).

15. A method for assessing coagulation defects or disorders in a subject comprising the steps of:
(aa) determining fibrinogen in a sample of said subject according to the method of claim 1;
(bb) comparing the determined fibrinogen to a reference; and
(cc) assessing coagulation defects or disorders in said subject based on said comparison.

16. A device for determining fibrinogen in a sample comprising:
(A) an analyzing unit comprising
(i) a reaction zone comprising a fibrinogen binding agent, said fibrinogen binding agent comprising a first molecule which is capable of specifically binding fibrinogen and, reversibly bound to the said first molecule, a second molecule which is capable of specifically binding the first molecule, wherein the affinity of said second molecule for the first molecule is lower than the affinity of fibrinogen for said first molecule, wherein said reaction zone is configured for contacting the fibrinogen binding agent with a sample suspected to comprise fibrinogen for a time and under conditions which allow for specific binding of fibrinogen to said first molecule whereby the second molecule is released from the first molecule and fibrinogen is specifically bound by the first molecule; and
(ii) a detector which is capable of determining the second molecule released from the fibrinogen binding agent;
and
(B) an evaluation unit which is capable of determining fibrinogen in the sample based on the released second molecule.

17. A kit for determining fibrinogen in a sample, comprising a fibrinogen binding agent, said fibrinogen binding agent comprising a first molecule which is capable of specifically binding fibrinogen and, reversibly bound to the said first molecule, a second molecule which is capable of specifically binding the first molecule, wherein the affinity of said second molecule for the first molecule is lower than the affinity of fibrinogen for said first molecule.
